(19) United States
(12) Patent Application Publication
(10) Pub. No.: US 2023/0248731 A1
(43) Pub. Date: Aug. 10, 2023

(54) COMPOSITION AND MEDICAMENT OF PALBOCICLIB ISETHIONATE

(71) Applicant: NANJING GRITPHARMA CO., LTD., Nanjing (CN)

(72) Inventors: Hongyan HE, Nanjing (CN); Jiannan Yang, Nanjing (CN); Yazhou He, Nanjing (CN); Yizhang Zuo, Nanjing (CN); Ruju Xiong, Nanjing (CN); Jinling Li, Nanjing (CN); Xiaotao Wu, Nanjing (CN); Liru Huo, Nanjing (CN); Zhan Li, Nanjing (CN)

(21) Appl. No.: 18/188,973

(22) Filed: Mar. 23, 2023

Related U.S. Application Data
(63) Continuation of application No. PCT/CN2021/119590, filed on Sep. 22, 2021.

(30) Foreign Application Priority Data
Sep. 24, 2020 (CN) 202011015214.8

(51) Int. Cl. A61K 31/519; A61K 9/16

(52) U.S. Cl. CPC A61K 31/519; A61K 9/1611; A61K 9/1623; A61K 9/1652

(57) ABSTRACT

The present invention discloses a composition of palbociclib isethionate and a medicament, and belongs to the technical field of pharmaceutical preparations. The composition includes palbociclib isethionate, a diluent, a disintegrant and a lubricant, as well as optionally a glidant. The palbociclib isethionate in the composition has a release rate of greater than or equal to 60% at a pH of 6.8 at 60 min. According to technical solutions of the present invention, influence of the pH environment in the human body on the release of a palbociclib isethionate medicament is reduced, the restriction that a medicament needs to be taken after a meal is overcome, and the medicament prepared from the composition of palbociclib isethionate can be taken before or after a meal without the restriction of achieving different effects when the medicament is taken before or after a meal.

### CROSS REFERENCE TO RELATED APPLICATIONS

This application is a continuation of International Application No. PCT/CN2021/119590, filed Sep. 22, 2021, entitled COMPOSITION AND DRUG OF PALBOCICLIB ISETHIONATE, which claims priority to Chinese Patent Application No. 202011015214.8 filed Sep. 24, 2020, which are incorporated by reference in their entirety.

### TECHNICAL FIELD

The present invention belongs to the technical field of pharmaceutical preparations, and more specifically relates to a composition and medicament of palbociclib isethionate.

### BACKGROUND

Breast cancer is the most common cancer in women. The breast cancer can be divided into an ER-positive type and an ER-negative type based on expression of an estrogen receptor (ER), and can also be divided into a Luminal A type, a Luminal B type, a basal-like type, an HER-2 overexpression type, a Normal-like type and other different molecular types based on analysis of genes. In the United States, two out of every 1,000 women are diagnosed with the breast cancer every year, and two-thirds of them are the estrogen receptor (ER)-positive type. ER-positive tumors are increased, while ER-negative tumors are decreased by 2% every year. Patients with ER-positive breast cancer generally have poor chemotherapy response. Thus, an adjuvant endocrinotherapy is recommended.

In the United States, palbociclib is indicated to treat postmenopausal patients with estrogen receptor-positive/ human epidermal growth factor receptor 2-negative (ER+/HER2-) advanced breast cancer in combination with letrozole to serve as an initial endocrinotherapy-based regimen for treatment of metastatic diseases, and was granted a “breakthrough therapy” certificate by the FDA of the U.S. in April 2013. Structures and preparation methods of the compound and salts thereof have already been described in a public document with an international application publication number of WO2003/062236 and a U.S. Pat. No. 6,936,612. Preparation methods of free alkalies and salts of various acids are also described in a public document with an international application publication number of WO2005/005426 and U.S. Pat. No. 7,345,171 and No. 7,863,278. It has been found in a medicine development process that solid properties of palbociclib isethionate are not good, and the uniformity is more difficult to control in a production scale environment. Thus, the palbociclib is produced for marketing in a free alkali form by Pfizer. However, the palbociclib in a free alkali form has poor solubility in water, resulting in low bioavailability and difficult absorption by the human body.

Oral bioavailability of a medicine refers to the absorption degree of the medicine in blood flow after oral administration.

The gastrointestinal tract consists of three parts including the stomach, small intestines and large intestines. Physiological conditions and medicine absorption conditions of the gastrointestinal tract are as shown in Table 1 below.

TABLE 1

Physiological conditions and medicine absorption conditions of the gastrointestinal tract

| Part | pH | Membrane | Blood flow | Surface area | Transit time |
|---|---|---|---|---|---|
| Stomach | 1-3 | Normal | Abundant | Small | 30-40 min |
| Small intestines (duodenum segment) | 6-6.5 | Normal | Abundant | Large | About 6s |
| Small intestines (jejunum and ileum segments) | 5-7 | Normal | Abundant | Large | 3-5 h |
| Large intestines | 5.5-7 | - | Abundant | Larger | As long as 24 h |

1.5-2.5 L of gastric juice is secreted by normal people every day, and the pH of the gastric juice in an empty stomach is 0.9-1.5, which is conducive to absorption of weakly acidic medicines. The pH of the gastric juice can be affected by water, food and diseases, and the secretion and pH of the gastric juice can also be affected by some medicines.

Most organic medicines are weakly alkaline or weakly acidic substances, and dissociation conditions of the medicines are affected by different changes in the pH of the digestive tract. Molecular medicines are easier to absorb than ionic medicines, so that absorption of the medicines is affected by the pH of secretions in the digestive tract.

Peristalsis of the gastrointestinal tract can make the food and medicines fully mixed, and has the effects of dispersing and stirring to make the medicines fully get contact with the gastric mucosa, so that convenience is provided for the absorption of the medicines in the stomach, and at the same time, contents are pushed forward to the duodenum.

When the contents in the stomach have low viscosity and osmotic pressure, the gastric emptying rate is generally large. When the viscosity and osmotic pressure of the contents are increased, the gastric emptying rate is decreased, and the gastric retention time is prolonged.

Factors of food affecting the absorption of medicines are as follows. 1. Water in the gastrointestinal tract is consumed, so that body fluids in the gastrointestinal tract are reduced, and disintegration of solid preparations and dissolution of medicines are slowed down. 2. The viscosity of contents in the gastrointestinal tract is increased, so that diffusion of the medicines to the wall of the gastrointestinal tract is hindered, and the absorption of the medicines is slowed down. 3. The gastric emptying time is prolonged. 4. The food, especially fat, can promote secretion of bile, so that the absorption of insoluble medicines is improved. 5. The pH of the gastrointestinal tract is changed, so that the absorption of weakly acidic and weakly alkaline medicines is affected. 6. Due to physical or chemical interaction with the medicines, the absorption is affected.

Existing clinical tests have also shown that the palbociclib isethionate has different bioavailability values before or after a meal. Pharmacokinetics of the palbociclib isethionate in healthy subjects after single administration were evaluated by Pfizer Pharmaceutical, an original research company. Summarized data of several major studies are as shown in Table 2 below. Different effects are achieved when the medicine is taken before or after a meal. Compared with administration before a meal, administration after a meal has the advantages that the Cmax value is increased by 7%, and the AUC_{inf} value is increased by about 9%. The food prolongs the retention time of the medicine in the gastric juice, so that the bioavailability is improved. The basic reason is that as the pH environment in the stomach and intestines is different, the palbociclib isethionate has inconsistent dissolution and release conditions, resulting in different bioavailability values. Therefore, when the dissolution of the palbociclib isethionate can be improved under neutral and weakly alkaline conditions, great benefits will be brought to application of the palbociclib isethionate to the treatment of related diseases in the human body.

TABLE 2

Pharmacokinetic parameters of palbociclib isethionate in healthy subjects after single oral administration in 125 mg

| Study number | Dosage form; fasting/eating | N | AUC_{inf} (ng·hr/mL) | Cₘₐₓ (ng/mL) | Tₘₐₓ (h) | T_{½} (h) | CL/F (L/h) | Vz/F (L) |
|---|---|---|---|---|---|---|---|---|
| Cancer patients | | | | | | | | |
| 1036 | Isethionate capsules; fasting overnight | 36 | 1468 (28) | 50.5 (33) | 6.0 (4.0-12.0) | 22.7 (±4.1) | 85.2 (28) | 2748 (28) |
| | Isethionate capsules; medium-fat meal a | 36 | 1596 (23) | 54.3 (25) | 6.0 (4.0-8.0) | 22.6 (±4.4) | 78.3 (23) | 2510 (22) |

AUC(₁₋₁₀) refers to an area under a concentration-time curve within 0-10 hours after administration; AUCᵢₙf refers to an area under a concentration-time curve; CL/F refers to apparent oral clearance; Cₘₐₓ refers to maximum plasma concentration; NC refers to no calculation; T_{½} refers to plasma half-life; Tmax refers to peak time; and Vz/F refers to apparent volume of distribution.
All PK parameters are geometric means (CV%), except that the Tmax is a median (range) and the T_{½} is an arithmetic mean (± standard deviation); and
^{a} indicates that administration is conducted 1 hour after a medium-fat meal is taken, and the medium-fat meal is taken again 2 hours after the administration.

### SUMMARY

### 1. Problems to Be Solved

In order to improve the bioavailability of palbociclib isethionate taken before a meal, the present invention provides a composition of palbociclib isethionate. By means of the composition, the dissolution of the palbociclib isethionate under neutral and weakly alkaline conditions can be equivalent to that under acidic conditions, so that influence of the pH environment in the human body on the release of a medicine is reduced, the restriction that a medicine needs to be taken before or after a meal is overcome, the palbociclib isethionate can be taken before or after a meal, the compliance is improved, and the restriction that different effects are achieved when the palbociclib isethionate is taken before or after a meal is overcome.

### 2. Technical Solutions

In order to solve the above problems, the applicant has studied the solubility of the palbociclib isethionate. As a pH-dependent medicament, the palbociclib isethionate has a good degree of dissociation in acidic gastric juice, and can be effectively absorbed. However, the palbociclib isethionate enters the intestinal tract rapidly on an empty stomach, and the pH environment becomes neutral and alkaline. As the solubility of the palbociclib isethionate is extremely low at a pH of 6.8, absorption of the palbociclib isethionate will be affected by the solubility, which is extremely unfavorable to the absorption and utilization of the palbociclib isethionate. In order to improve the dissolution of the palbociclib isethionate under neutral and alkaline conditions, the following technical solutions are adopted by the present invention.

A composition of palbociclib isethionate includes the following components in parts by mass:

| | |
|---|---|
| palbociclib isethionate | 25-50 parts |
| diluent | 40-70 parts |
| disintegrant | 1-15 parts |
| glidant | 0-10 parts |
| lubricant | 0.1-10 parts. |

Preferably, when the composition is tested in 900 mL of a phosphate buffer medium with a pH of 6.8 at 50 rpm and a dissolution temperature of 37±0.5° C. by a paddle method, the palbociclib isethionate has a release rate of greater than or equal to 60% at 60 min. It should be noted that investigation of the release degree index in the present invention includes investigation of the release degree of the composition or any preparation form prepared from the composition, such as tablets and capsules. When the composition filled in a capsule form is tested in 900 mL of a phosphate buffer medium with a pH of 6.8 at 50 rpm and a dissolution temperature of 37±0.5° C. by a paddle method, the palbociclib isethionate has a release rate of greater than or equal to 60% at 60 min. Undoubtedly, when the same test is carried out on the composition in capsules directly, the palbociclib isethionate also has a release rate of greater than or equal to 60% at 60 min.

Preferably, when the composition is tested in 900 mL of a hydrochloric acid medium with a pH of 1.2 at 50 rpm and a dissolution temperature of 37±0.5° C. by a paddle method, the palbociclib isethionate has a release rate of greater than or equal to 85% at 15 min.

Studies have found that in vitro release behaviors of a product are affected by the particle size of the palbociclib isethionate. When the particle size is decreased, in vitro release of the palbociclib isethionate in the composition is accelerated at a pH of 6.8, and in vivo absorption is also affected by the in vitro release behaviors. Therefore, further, a raw material of the palbociclib isethionate has a particle size D90 of 3-60 µm, preferably 3-30 µm, and further preferably 3-20 µm.

Further, the particle size of the palbociclib isethionate can be controlled in a synthesis process of the raw material of the palbociclib isethionate, or the particle size of the palbociclib isethionate is controlled by conducting a pulverization, grinding or micropulverization process on the raw material of the palbociclib isethionate prepared.

Further, the particle size of the palbociclib isethionate is preferably controlled in a synthesis process of the raw material of the palbociclib isethionate.

Further, the composition preferably includes 30-45 parts by mass of the palbociclib isethionate.

Further, the diluent is selected from one or more of lactose, microcrystalline cellulose, pregelatinized starch, mannitol or calcium hydrogen phosphate.

Further, the composition includes 40-70 parts by mass of the diluent, preferably 50-60 parts by mass.

Further, the disintegrant is selected from one or more of crospovidone, sodium carboxymethyl starch, crosslinked sodium carboxymethyl cellulose, crosslinked calcium carboxymethyl cellulose or low-substituted hydroxypropyl cellulose for combined use.

Further, the composition includes 1-15 parts by mass of the disintegrant, preferably 3-10 parts by mass.

Further, the glidant is optionally selected from one or more of silicon dioxide, talc powder or polyethylene glycol, and is preferably silicon dioxide.

Further, the composition includes 0-10 parts by mass of the glidant, preferably 0.5-5 parts by mass.

Further, the lubricant is selected from one or more of magnesium stearate, sodium stearyl fumarate, calcium stearate or stearic acid, and is preferably magnesium stearate.

Further, the composition includes 0.1-10 parts by mass of the lubricant, preferably 0.5-4 parts by mass.

Further, a mass ratio of the palbociclib isethionate to the diluent during premixing is 1: (0.8-2.0).

A medicament includes the composition of palbociclib isethionate. Granules obtained after granulation of the composition have a tap density of 0.55-0.72 g/mL and an angle of repose of less than or equal to 44°.

Further, the tap density is 0.62-0.69 g/mL.

### 3. Beneficial Effects

Compared with the prior art, the present invention has the following beneficial effects.

When the composition of palbociclib isethionate in the present invention is tested in 900 mL of a phosphate buffer medium with a pH of 6.8 at 50 rpm and a dissolution temperature of 37±0.5° C. by a paddle method, the palbociclib isethionate has a release rate of greater than or equal to 60% at 60 min, which is equivalent to that at a pH of 1.2, so that influence of the pH environment in the human body on the release of the medicine is reduced, the restriction that the medicine needs to be taken after a meal is overcome, the medicament prepared from the composition of palbociclib isethionate can be taken before or after a meal without the restriction of achieving different effects when the medicament is taken before or after a meal, and the compliance is improved.

Studies have found that in vitro release behaviors of a product are affected by the particle size of the palbociclib isethionate. When the particle size is decreased, for example, when the particle size D90 of the palbociclib isethionate in the composition is in the range of 3-60 µm, in vitro release of the palbociclib isethionate is accelerated at a pH of 6.8, and the release rate is 60% or above at 60 min.

According to the composition with certain components and ratios formed by palbociclib isethionate, a diluent, a disintegrant and a lubricant, as well as optionally a glidant in the present invention, it can be ensured that the release rate of the palbociclib isethionate is 60% or above at 60 min at a pH of 6.8.

The palbociclib isethionate has poor fluidity and is a loose powder. When capsules are filled, especially when capsules are filled in commercial batches in a large scale, the problem of difficulty in automatic filling at high speed will be caused due to poor fluidity. Studies have found that the problem can be better solved by conducting granulation on palbociclib isethionate and pharmaceutically acceptable auxiliary materials and controlling granules obtained after the granulation to have a tap density of 0.55-0.72 g/mL and an angle of repose of less than or equal to 44°. The tap density of the granules obtained after the granulation is further preferably in the range of 0.62-0.69 g/mL, and a composition sample which has good fluidity and is easy to fill and process can be obtained.

### BRIEF DESCRIPTION OF THE DRAWINGS

FIG. 1 shows release curves of palbociclib isethionate capsules prepared from raw materials different in particle size of palbociclib isethionate in Example 1 and palbociclib capsules in a free alkali form in a medium with a pH of 1.2;

FIG. 2 shows release curves of palbociclib isethionate capsules prepared from raw materials different in particle size of palbociclib isethionate in Example 1 and palbociclib capsules in a free alkali form in a medium with a pH of 6.8;

FIG. 3 shows release curves of palbociclib isethionate capsules prepared from raw materials different in particle size of palbociclib isethionate in Example 2 and palbociclib capsules in a free alkali form in a medium with a pH of 1.2;

FIG. 4 shows release curves of palbociclib isethionate capsules prepared from raw materials different in particle size of palbociclib isethionate in Example 2 and palbociclib capsules in a free alkali form in a medium with a pH of 6.8;

FIG. 5 shows release curves of 5 batches of samples of palbociclib isethionate capsules in Example 9 in a medium with a pH of 1.2; and

FIG. 6 shows release curves of 5 batches of samples of palbociclib isethionate capsules in Example 9 in a medium with a pH of 6.8.

### DETAILED DESCRIPTION

Unless otherwise defined, meanings of all technical and scientific terms used herein are the same as those usually understood by a person skilled in the art to which the present invention belongs. The term “and/or” used herein includes any or all combinations of one or more of related listed items.

When specific conditions are not specified in embodiments, processes shall be carried out in accordance with conventional conditions or conditions suggested by manufactures. All reagents or instruments used without specific manufacturers are commercially available conventional products.

As used herein, the term “about” is used for providing flexibility and imprecision associated with a given term, measure, or value. The degree of flexibility of specific variables can be easily determined by a person skilled in the art.

Parts, concentrations, quantities, and other numerical data can be presented in the form of a range herein. It should be understood that the form of a range is used only for convenience and brevity, and should be interpreted flexibly to include not only values that are explicitly stated as limits of the range, but also all individual values or subranges that are covered within the range, as if each value and subrange are explicitly stated. For example, the numerical range from about 1 to about 4.5 should be interpreted to include not only explicitly stated limits from 1 to about 4.5, but also individual numbers (such as 2, 3, and 4) and subranges (such as 1 to 3 and 2 to 4). The same principle is applicable to ranges that state only one value, such as “less than about 4.5,” which should be interpreted to include all of the above values and ranges. In addition, the interpretation should apply regardless of the scope or breadth of features described.

Any steps described in any method or process claims may be carried out in any order, and are not limited to the order set forth in the claims. Limitations of a method and a function or a step and a function are adopted only when all of the following conditions are satisfied in a specificclaim limitation: a) a “method for......” or a “step for......” is explicitly stated; and b) corresponding functions are explicitly stated. Therefore, the scope of the present invention shall be determined only by the attached claims and legal equivalents thereof and not by the description and embodiments provided herein.

The present invention is further described below in conjunction with specific embodiments.

First, the applicant has studied the solubility of palbociclib isethionate in water, a hydrochloric acid solution with a pH of 1.0, a phosphate buffer with a pH of 6.8 and an acetate buffer with a pH of 4.5 (as shown in Table 3). It has been found that the palbociclib isethionate is a pH-dependent medicament, has a good degree of dissociation in acidic gastric juice, and can be effectively absorbed. However, the palbociclib isethionate enters the intestinal tract rapidly on an empty stomach, and the pH environment becomes neutral and weakly alkaline. As the solubility of the palbociclib isethionate is extremely low at a pH of 6.8, absorption of the palbociclib isethionate will be affected by the solubility, which is extremely unfavorable to the absorption and utilization of the palbociclib isethionate.

TABLE 3

Solubility data of palbociclib isethionate at different pH values

| Serial number | pH | Solubility (mg/mL) |
|---|---|---|
| 1 | 1.0 | 90.7 |
| 2 | 4.5 | 84.7 |
| 3 | 6.8 | 0.04 |
| 4 | Water | 84.8 |

### Example 1

A prescription includes the following components:

| | |
|---|---|
| Palbociclib isethionate | 160.23 g |
| Lactose | 82.7 g |
| Microcrystalline cellulose | 165.4 g |
| Crospovidone | 27 g |
| Colloidal silicon dioxide | 5.7 g |
| Magnesium stearate | 9 g |
| Prepared into | 1,000 granules |

### Preparation Process:

In this example, in order to investigate differences of in vitro release behaviors of products caused by the particle size, raw materials of palbociclib isethionate with the following particle size D90 were separately used: 3 µm, 20 µm, 30 µm, 60 µm, 80 µm, and 120 µm (the particle size of the raw materials was tested by a laser particle size analyzer of Sympatec and a particle size tester with a model of HELOS/BR-RODOS/T4&CUVETTE; and test parameters were as follows: a dispersion system of RODOD/T4 was selected, an R5 (0.5-875 µm) lens was selected, an injector was set to VIBRI, the injection rate was 20%-90%, the dispersion pressure was 3.5-4.5 bar, and the parameters were the same below). The palbociclib isethionate different in particle size was obtained by synthesis. Capsules were prepared by the following method according to the components in the above prescription.

### 1. Preparation of Raw and Auxiliary Materials

The raw materials of palbociclib isethionate (with the particle size D90 of 3 µm, 20 µm, 30 µm, 60 µm, 80 µm, and 120 µm separately), the microcrystalline cellulose and the crospovidone were separately sifted with a 40-mesh sieve, and the lactose, the colloidal silicon dioxide and the magnesium stearate were separately sifted with an 80-mesh sieve for later use.

### 2. Premixing

The palbociclib isethionate and the microcrystalline cellulose were weighed according to the amounts in the prescription respectively, premixed, and then sifted with a 40-mesh sieve. A premix, the lactose, the crospovidone and the colloidal silicon dioxide were added to a three-dimensional motion mixer and mixed for 15 min.

### 3. Dry Granulation

After parameters of a dry granulation machine were set, a mixture obtained in step 2 was subjected to dry granulation.

### 4. Blending

The magnesium stearate and granules obtained after the dry granulation were added to the three-dimensional motion mixer and blended for uniform mixing for 10 min.

### 5. Filling of Capsules

Capsules were filled with blended granules by using a capsule filling machine, where 0# capsules (hollow gelatin capsules produced by the manufacturer Suzhou Capsule Capsugel® LTD. were used in the example) were used, and the filling capacity was 450 mg.

Obtained samples were recorded as a sample 1-1 (the particle size D90 of the raw material of palbociclib isethionate was 3 µm), a sample 1-2 (the particle size D90 of the raw material of palbociclib isethionate was 20 µm), a sample 1-3 (the particle size D90 of the raw material of palbociclib isethionate was 30 µm), a sample 1-4 (the particle size D90 of the raw material of palbociclib isethionate was 60 µm), a sample 1-5 (the particle size D90 of the raw material of palbociclib isethionate was 80 µm), and a sample 1-6 (the particle size D90 of the raw material of palbociclib isethionate was 120 µm).

### Example 2

A prescription includes the following components:

| | |
|---|---|
| Palbociclib isethionate | 128.18 g |
| Lactose | 66.16 g |
| Microcrystalline cellulose | 132.32 g |
| Crospovidone | 21.6 g |
| Colloidal silicon dioxide | 4.56 g |
| Magnesium stearate | 7.2 g |
| Prepared into | 1,000 granules |

### Preparation Process:

In this example, in order to investigate differences of in vitro release behaviors of products caused by the particle size, raw materials of palbociclib isethionate with the following particle size D90 were separately used: 3 µm, 20 µm, and 60 µm. The raw material with the particle size D90 of 3 µm was obtained by air flow pulverization, and the raw materials with the particle size D90 of 20 µm and 60 µm were separately obtained by synthesis. Capsules were prepared by the following method according to the components in the above prescription under three conditions.

### 1. Preparation of Raw and Auxiliary Materials

The raw materials of palbociclib isethionate, the microcrystalline cellulose and the crospovidone were separately sifted with a 40-mesh sieve, and the lactose, the colloidal silicon dioxide and the magnesium stearate were separately sifted with an 80-mesh sieve for later use.

### 2. Premixing

The palbociclib isethionate and the microcrystalline cellulose were weighed according to the amounts in the prescription respectively, premixed, and then sifted with a 40-mesh sieve. A premix, the lactose, the crospovidone and the colloidal silicon dioxide were added to a three-dimensional motion mixer and mixed for 15 min.

### 3. Dry Granulation

After parameters of a dry granulation machine were set, a mixture obtained in step 2 was subjected to dry granulation.

### 4. Blending

The magnesium stearate and granules obtained after the dry granulation were added to the three-dimensional motion mixer and blended for uniform mixing for 10 min.

### 5. Filling of Capsules

Capsules were filled with blended granules by using a capsule filling machine, where 1# capsules (hollow gelatin capsules produced by the manufacturer Suzhou Capsule Capsugel (R) LTD. were used in the example) were used, and the filling capacity was 360 mg.

Obtained samples were recorded as a sample 2-1 (the particle size D90 of the raw material of palbociclib isethionate was 3 µm), a sample 2-2 (the particle size D90 of the raw material of palbociclib isethionate was 20 µm), and a sample 2-3 (the particle size D90 of the raw material of palbociclib isethionate was 60 µm).

The samples prepared from raw materials different in particle size in Example 1 and Example 2 were separately sampled at different time points to investigate the release rate in 900 mL of a hydrochloric acid medium with a pH of 1.2 at 50 rpm and 900 mL of a phosphate buffer medium with a pH of 6.8 at 50 rpm by a paddle method, so as to study in vitro release behaviors.

Test results of the release rate of products prepared from raw materials different in particle size show the following conclusions.

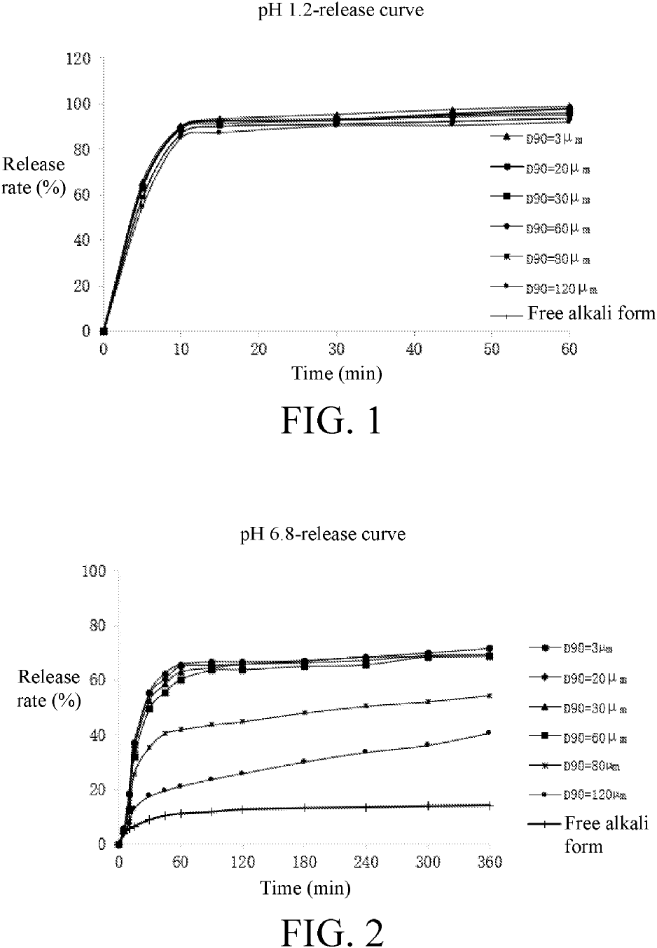

The capsule samples prepared from raw materials different in particle size in Example 1 of the present invention and palbociclib isethionate capsules in a free alkali form (commercially available product, Ibrance® with a specification of 125 mg) produced by Pfizer were taken to study in vitro release behaviors, and have release curves as shown in FIG. 1 at a pH of 1.2 (as capsule shells of the same type were used, influence of the capsule shells on the release rate can be excluded). Results show that the samples prepared from raw materials different in particle size have release behaviors basically consistent with that of the palbociclib isethionate capsules in a free alkali form in the medium with a pH of 1.2, and the dissolution rate is greater than 85% at 15 min. However, as shown in FIG. 2, the release rate of the palbociclib isethionate capsules in a free alkali form (commercially available product, Ibrance® with a specification of 125 mg) produced by Pfizer is still less than 20% at 360 min in the medium with a pH of 6.8, which is the same as that in previous studies. The palbociclib isethionate capsules in a free alkali form are suitable for taking after a meal, and have poor bioavailability when taking before a meal. On the contrary, the release rate of the capsules of the compositions prepared from the raw materials of palbociclib isethionate with the D90 of 3-120 µm is greater than that of the palbociclib isethionate capsules in a free alkali form in the medium with a pH of 6.8. When the raw materials of palbociclib isethionate different in particle size are used, the release behaviors are different. With the increase of the particle size, the release is slowed down. The capsules of the compositions prepared from the raw materials of palbociclib isethionate with the D90 of 3-60 µm have a basically consistent release rate at 60 min, and the release rate is greater than 60%.

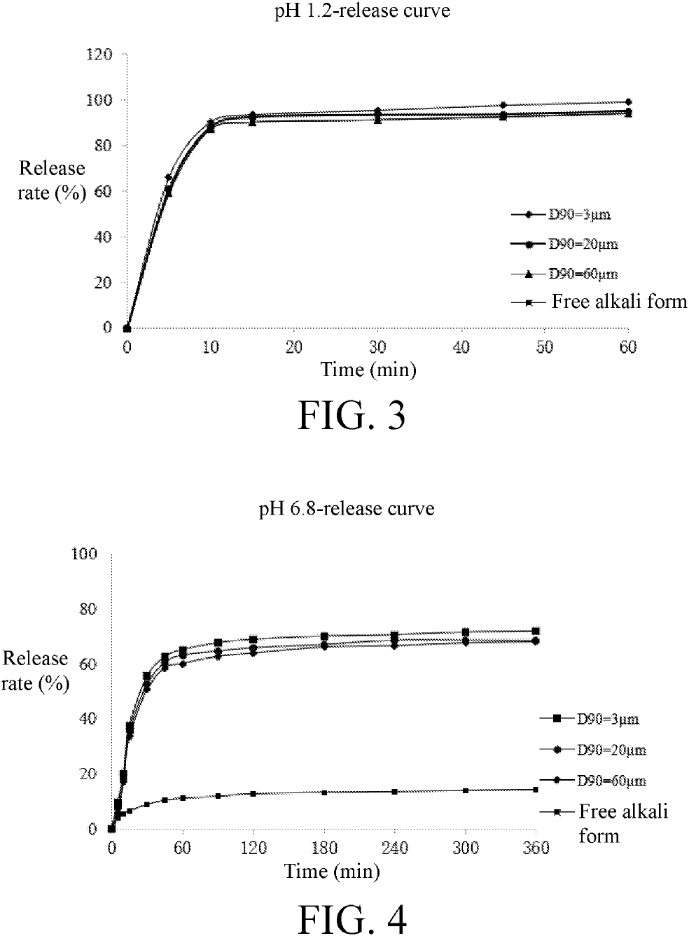

The capsule samples prepared from raw materials different in particle size in Example 2 of the present invention were taken to study in vitro release behaviors, and have release curves as shown in FIG. 3 at a pH of 1.2. Results show that the capsule samples prepared from raw materials different in particle size have release behaviors basically consistent with that of the palbociclib isethionate capsules in a free alkali form in the medium with a pH of 1.2, and the release rate is greater than 85% at 15 min. However, as shown in FIG. 4, the release rate of the palbociclib isethionate capsules in a free alkali form (commercially available product, Ibrance® with a specification of 125 mg) produced by Pfizer is still less than 20% at 360 min in the medium with a pH of 6.8. On the contrary, the release rate of the capsules of the compositions prepared from the raw materials of palbociclib isethionate with the D90 of 3-60 µm is greater than that of the palbociclib isethionate capsules in a free alkali form in the medium with a pH of 6.8. When the raw materials of palbociclib isethionate different in particle size are used, the release behaviors are basically consistent, and the release rate is greater than 60% at 60 min. Therefore, when the particle size of the raw materials of palbociclib isethionate is controlled to 3-60 µm, the effect of good bioavailability can also be achieved when the palbociclib isethionate is taken before a meal. When the particle size D90 of the raw materials of palbociclib isethionate is 3-20 µm, the release rate is high, and thus this range is a more preferred particle size range.

Through comparison between Example 1 (palbociclib isethionate with a specification of 160.23 mg) and Example 2 (palbociclib isethionate with a specification of 128.18 mg), different specifications of the palbociclib isethionate have basically the same release rate at a pH of 1.2 and a pH of 6.8.

### Example 3

A prescription includes the following components:

| | |
|---|---|
| Palbociclib isethionate | 96.14 g |
| Lactose | 42.7 g |
| Microcrystalline cellulose | 105.4 g |
| Crospovidone | 15 g |
| Colloidal silicon dioxide | 6.76 g |
| Magnesium stearate | 4 g |
| Prepared into | 1,000 granules |

### 1. Preparation of Raw and Auxiliary Materials

A raw material of the palbociclib isethionate (D90 was 31 µm), the microcrystalline cellulose and the crospovidone were separately sifted with a 40-mesh sieve, and the lactose, the colloidal silicon dioxide and the magnesium stearate were separately sifted with an 80-mesh sieve for later use.

### 2. Premixing

The palbociclib isethionate and the microcrystalline cellulose were weighed according to the amounts in the prescription respectively, premixed, and then sifted with a 40-mesh sieve. A premix, the lactose, the crospovidone and the colloidal silicon dioxide were added to a three-dimensional motion mixer and mixed for 15 min.

### 3. Dry Granulation

After parameters of a dry granulation machine were set, a mixture obtained in step 2 was subjected to dry granulation.

### 4. Blending

The magnesium stearate and granules obtained after the dry granulation were added to the three-dimensional motion mixer and blended for uniform mixing for 10 min.

### 5. Filling of Capsules

Capsules were filled with blended granules by using a capsule filling machine, where 2# capsules (hollow gelatin capsules produced by the manufacturer Suzhou Capsule Capsugel® LTD. were used in the example) were used, and the filling capacity was 270 mg.

The release rate of the capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 4

A prescription includes the following components:

| | |
|---|---|
| Palbociclib isethionate (D90 was 45 µm) | 160.23 g |
| Lactose | 52.7 g |
| Microcrystalline cellulose | 225.4 g |
| Sodium carboxymethyl starch | 26.67 g |
| Colloidal silicon dioxide | 10 g |
| Magnesium stearate | 9 g |
| Prepared into | 1,000 granules |

A specific preparation process and a capsule specification were the same as those in Example 1.

The release rate of the capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 5

A prescription includes the following components:

| | |
|---|---|
| Palbociclib isethionate (D90 was 15 µm) | 128.18 g |
| Calcium hydrogen phosphate | 25.16 g |
| Microcrystalline cellulose | 162.32 g |
| Crospovidone | 31.6 g |
| Sodium stearyl fumarate | 6.2 g |
| Prepared into | 1,000 granules |

A specific preparation process and a capsule specification were the same as those in Example 2.

The release rate of the capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 6

A prescription includes the following components:

| | |
|---|---|
| Palbociclib isethionate (D90 was 50 µm) | 96.14 g |
| Lactose | 32 g |
| Microcrystalline cellulose | 103 g |
| Crospovidone | 27 g |
| Colloidal silicon dioxide | 13 g |
| Magnesium stearate | 2 g |
| Prepared into | 1,000 granules |

A specific preparation process and a capsule specification were the same as those in Example 3.

The release rate of the capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 7

A prescription includes the following components:

| | |
|---|---|
| Palbociclib isethionate (D90 was 25 µm) | 96.14 g |
| Lactose | 82.7 g |
| Microcrystalline cellulose | 165.4 g |
| Crospovidone | 27 g |
| Colloidal silicon dioxide | 5.7 g |
| Magnesium stearate | 9 g |
| Prepared into | 1,000 granules |

A specific preparation process was the same as that in Example 1. 1# capsules (hollow gelatin capsules produced by the manufacturer Suzhou Capsugel ® LTD. were used in the example) were used, and the filling capacity was 386 mg.

The release rate of capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 8

A prescription includes the following components:

| | |
|---|---|
| Palbociclib isethionate (D90 was 32 µm) | 128.18 g |
| Lactose | 30.1 g |
| Microcrystalline cellulose | 75.28 g |
| Crospovidone | 16 g |
| Colloidal silicon dioxide | 3.8 g |
| Magnesium stearate | 3 g |
| Prepared into | 1,000 granules |

A specific preparation process was the same as that in Example 1. 2# capsules (hollow gelatin capsules produced by the manufacturer Suzhou Capsugel ® LTD. were used in the example) were used, and the filling capacity was 256 mg.

The release rate of capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

### Example 9

A prescription includes the following components:

| | |
|---|---|
| Palbociclib isethionate (D90 was 20 µm) | 128.18 g |
| Calcium hydrogen phosphate | 96.16 g |
| Microcrystalline cellulose | 102.32 g |
| Calcium carboxymethyl cellulose | 21.6 g |
| Colloidal silicon dioxide | 4.56 g |
| Magnesium stearate | 7.2 g |
| Prepared into | 1,000 granules |

### Preparation Process:

### 1. Preparation of Raw and Auxiliary Materials

The palbociclib isethionate, the microcrystalline cellulose and the crospovidone were sifted with a 40-mesh sieve, and the calcium hydrogen phosphate, the colloidal silicon dioxide and the magnesium stearate were sifted with an 80-mesh sieve for later use.

### 2. Premixing

The palbociclib isethionate and the microcrystalline cellulose were weighed according to the amounts in the prescription respectively, premixed, and then sifted with a 40-mesh sieve.

### 3. Mixing

A premix, the calcium hydrogen phosphate and the calcium carboxymethyl cellulose were added to a three-dimensional motion mixer and mixed for 15 min.

### 4. Dry Granulation

After parameters of a dry granulation machine were set, a mixture obtained in step 3 was subjected to dry granulation. The press roll pressure was set to 25-35 kg/cm³, 35-45 kg/cm³, 45-55 kg/cm³, 55-65 kg/cm³, and 65-70 kg/cm³ separately to obtain five batches of dry granulated samples.

### 5. Blending

The colloidal silicon dioxide, the magnesium stearate and three batches of granules obtained after the dry granulation were separately added to the three-dimensional motion mixer and blended for uniform mixing for 10 min.

### 6. Filling of Capsules

Capsules were filled with blended granules by using a capsule filling machine, where 1# capsules (hollow gelatin capsules produced by the manufacturer Suzhou Capsule Capsugel® LTD. were used in the example) were used, and the filling capacity was 360 mg.

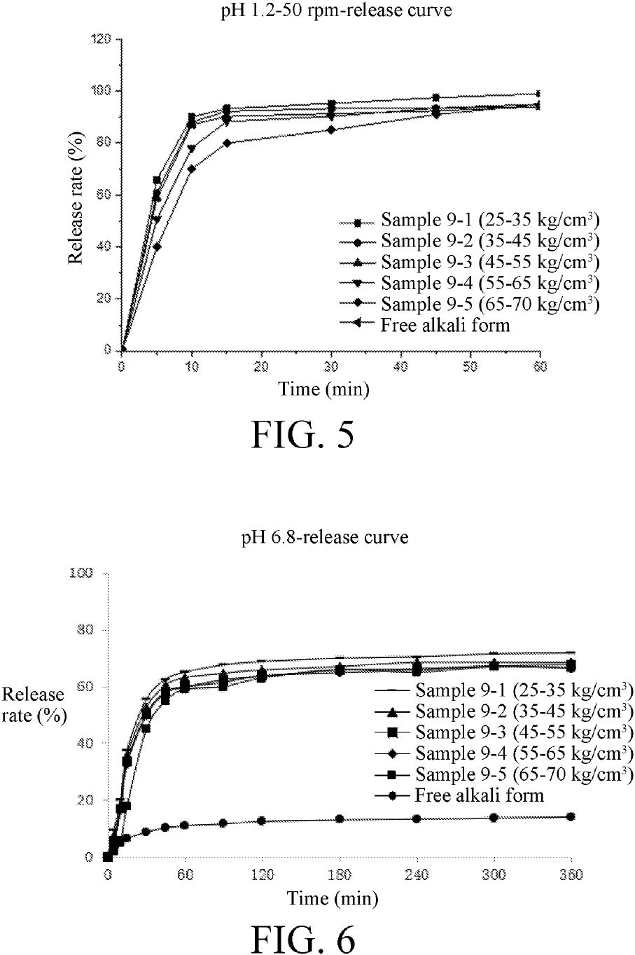

5 batches of samples 9-1, 9-2, 9-3, 9-4, and 9-5 were prepared by a capsule filling process after granules were prepared by dry granulation at different press roll pressures, and properties of the granules (including angle of repose, bulk density, and tap density), content uniformity of finished products, and other indexes were investigated separately. Results show that the five samples prepared by the dry granulation at different press roll pressures can be better filled. According to requirements of pharmacopoeia, the release rate of the samples is greater than or equal to 80% (Q) of the labelled amount in a medium with a pH of 1.2 within 30 minutes. Thus, the release rate of the various batches of samples (9-1, 9-2, 9-3, 9-4, and 9-5) was investigated in a medium with a pH of 1.2. Results show that the release of all the samples is qualified. Results are as shown in Table 4 and FIG. 5 below.

TABLE 4

| | | | | | |
|---|---|---|---|---|---|
| Sample | Sample 9-1 | Sample 9-2 | Sample 9-3 | Sample 9-4 | Sample 9-5 |
| Press roll pressure (kg/cm³) | 25-35 | 35-45 | 45-55 | 55-65 | 65-70 |
| Powder mix angle of repose (°) | 38 | 36 | 37 | 37 | 38 |
| Bulk density (g/cm³) | 0.530 | 0.578 | 0.621 | 0.640 | 0.650 |
| Tap density (g/cm³) | 0.620 | 0.650 | 0.690 | 0.720 | 0.741 |
| Compression index | 14.5 | 11.1 | 10.0 | | |
| Filling property | Capsules have good filling conditions | Capsules have good filling conditions | Capsules have good filling conditions | Capsules have good filling conditions | Capsules have good filling conditions |
| Loading difference (limit requirement ± 7.5%) | -2.3% to 4.7% | -3.0% to 2.1% | -1.3% to 2.9% | -1.0% to 1.2% | -1.2% to 1.1% |
| Content uniformity (limit requirement A+2.2S≤15) | 6.1 | 5.7 | 2.7 | 2.4 | 2.5 |
| Disintegration time (min) | 2 min 50 s | 3 min 30 s | 5 min 46 s | 10 min | 16 min |

The above study data indicate that the prescription powder mix of the present invention has good compressibility in dry granulation at a press roll pressure of 25-65 kg/cm³, a powder mix angle of repose of 36-38° and a tap density of 0.62-0.72 g/cm³, the granular powder mix prepared has good fluidity and can be easily filled into capsules, a filling process is stable, filled capsules have small differences, the content uniformity of the samples meets requirements of a content uniformity test method of General Rule 0941 of Pharmacopoeia (2015 edition), and the disintegration time is shorter than 15 min. With further increase of the pressure, the disintegration time is prolonged, resulting in decrease of the dissolution rate.

FIG. 5 shows that capsules are filled with granules prepared from the prescription powder mix of the present invention by dry granulation at a press roll pressure of 25-65 kg/cm³, and the prepared samples have in vitro release behaviors basically consistent with that of capsules in a free alkali form in a medium with a pH of 1.2. As shown in FIG. 6, release curves of the five samples from 9-1 to 9-5 at a pH of 6.8 indicate that the samples from 9-1 to 9-5 have a release rate of greater than 60% at 60 min at a pH of 6.8, and have a higher release rate than the capsules in a free alkali form.

Therefore, when the press roll pressure is controlled in the range of 25-65 kg/cm³ in the dry granulation process with the same dry granulation machine as that in the example, samples which have good fluidity, are easily filled into capsules, and have a release rate of greater than 60% at 60 min at a pH of 6.8 can be obtained.

### Example 10

A prescription includes the following components:

| | |
|---|---|
| Palbociclib isethionate (D90 was 30 µm) | 160.23 g |
| Lactose | 82.7 g |
| Microcrystalline cellulose | 165.4 g |
| Crospovidone | 27 g |
| Colloidal silicon dioxide | 5.7 g |
| Magnesium stearate | 9 g |
| Prepared into | 1,000 granules |

### Preparation Process:

### 1. Preparation of Raw and Auxiliary Materials

The palbociclib isethionate, the microcrystalline cellulose and the crospovidone were sifted with a 40-mesh sieve, and the lactose, the colloidal silicon dioxide and the magnesium stearate were sifted with an 80-mesh sieve for later use.

### 2. Mixing

The palbociclib isethionate, the microcrystalline cellulose, the lactose, the crospovidone and the colloidal silicon dioxide were weighed according to the amounts in the prescription respectively, added to a three-dimensional motion mixer and mixed for 15 min.

### 3. Dry Granulation

After parameters of a dry granulation machine were set, a mixture obtained in step 2 was subjected to dry granulation.

### 4. Blending

The magnesium stearate and granules obtained after the dry granulation were added to the three-dimensional motion mixer and blended for uniform mixing for 10 min.

### 5. Filling of Capsules

Capsules were filled with blended granules by using a capsule filling machine, where 0# capsules (hollow gelatin capsules produced by the manufacturer Suzhou Capsule Capsugel® LTD. were used in the example) were used, and the filling capacity was 450 mg.

The granules prepared by using the method in this example have a powder mix angle of repose of 44°, a bulk density of 0.509 g/cm³, a tap density of 0.567 g/cm³, a loading difference (limit requirement ± 7.5%) of -5.5% to 5.9%, a content uniformity (limit requirement A+2.2S≤15) of 14.3, and good fluidity. The capsules can be better filled.

The release rate of the capsules in this example was separately tested by the same release rate measurement method as above at a pH of 1.2 and a pH of 6.8. Results show that the capsules prepared in this example have release behaviors basically consistent with that of capsules in a free alkali form at a pH of 1.2, but the release rate is greater than that of the capsules in a free alkali form at a pH of 6.8, and the release rate is greater than 60% at 60 min.

The above content is a schematic description of the present invention and embodiments thereof, and the description is not restrictive. The content as shown in the examples is only one of the embodiments of the present invention, and the embodiments are actually not limited thereto. Therefore, when those of ordinary skill in the art are inspired by the present invention, structural modes and examples made similar to the technical solutions without creative design on the premise of not departing from the creative purpose of the present invention shall fall within the protection scope of the present invention.

What is claimed is:

1. A composition of palbociclib isethionate, comprising the following components in parts by mass:

| | |
|---|---|
| palbociclib isethionate | 25-50 parts |
| diluent | 40-70 parts |
| disintegrant | 1-15 parts |
| glidant | 0-10 parts |
| lubricant | 0.1-10 parts |

.

2. The composition of palbociclib isethionate according to claim 1, wherein when the composition is tested in 900 mL of a phosphate buffer medium with a pH of 6.8 at 50 rpm and a dissolution temperature of 37±0.5° C. by a paddle method, the palbociclib isethionate has a release rate of greater than or equal to 60% at 60 min.

3. The composition of palbociclib isethionate according to claim 1, wherein when the composition is tested in 900 mL of a hydrochloric acid medium with a pH of 1.2 at 50 rpm and a dissolution temperature of 37±0.5° C. by a paddle method, the palbociclib isethionate has a release rate of greater than or equal to 85% at 15 min.

4. The composition of palbociclib isethionate according to claim 1, wherein a raw material of the palbociclib isethionate has a particle size D90 of 3-60 µm.

5. The composition of palbociclib isethionate according to claim 4, wherein a raw material of the palbociclib isethionate has a particle size D90 of 3-20 µm.

6. The composition of palbociclib isethionate according to claim 4, wherein the particle size of the palbociclib isethionate is controlled in a synthesis process of the raw material of the palbociclib isethionate, or the particle size of the palbociclib isethionate is controlled by conducting a pulverization, grinding or micropulverization process on the raw material of the palbociclib isethionate prepared.

7. The composition of palbociclib isethionate according to claim 4, wherein the particle size of the palbociclib isethionate is controlled in a synthesis process of the raw material of the palbociclib isethionate.

8. The composition of palbociclib isethionate according to claim 1, wherein the composition comprises 30-45 parts by mass of the palbociclib isethionate.

9. The composition of palbociclib isethionate according to claim 1, wherein the diluent is selected from one or more of lactose, microcrystalline cellulose, pregelatinized starch, mannitol or calcium hydrogen phosphate.

10. The composition of palbociclib isethionate according to claim 9, wherein the composition comprises 50-60 parts by mass of the diluent.

11. The composition of palbociclib isethionate according to claim 9, wherein the disintegrant is selected from one or more of crospovidone, sodium carboxymethyl starch, crosslinked sodium carboxymethyl cellulose, crosslinked calcium carboxymethyl cellulose or low-substituted hydroxypropyl cellulose for combined use.

12. The composition of palbociclib isethionate according to claim 11, wherein the composition comprises 3-10 parts by mass of the disintegrant.

13. The composition of palbociclib isethionate according to claim 11, wherein the glidant is optionally selected from one or more of silicon dioxide, talc powder or polyethylene glycol.

14. The composition of palbociclib isethionate according to claim 13, wherein the glidant is silicon dioxide.

15. The composition of palbociclib isethionate according to claim 13, wherein the composition comprises 0.5-5 parts by mass of the glidant.

16. The composition of palbociclib isethionate according to claim 13, wherein the lubricant is selected from one or more of magnesium stearate, sodium stearyl fumarate, calcium stearate or stearic acid.

17. The composition of palbociclib isethionate according to claim 16, wherein the lubricant is magnesium stearate.

18. The composition of palbociclib isethionate according to claim 16, wherein the composition comprises 0.5-4 parts by mass of the lubricant.

19. A medicament, comprising the composition of palbociclib isethionate according to claim 1, wherein granules obtained after granulation of the composition have a tap density of 0.55-0.72 g/mL and an angle of repose of less than or equal to 44°.

20. The medicament according to claim 19, wherein the tap density is 0.62-0.69 g/mL.

21. A medicament, comprising the composition of palbociclib isethionate according to claim 2, wherein granules obtained after granulation of the composition have a tap density of 0.55-0.72 g/mL and an angle of repose of less than or equal to 44°.

22. The composition of palbociclib isethionate according to claim 2, wherein when the composition is tested in 900 mL of a hydrochloric acid medium with a pH of 1.2 at 50 rpm and a dissolution temperature of 37±0.5° C. by a paddle method, the palbociclib isethionate has a release rate of greater than or equal to 85% at 15 min.

23. The composition of palbociclib isethionate according to claim 2, wherein a raw material of the palbociclib isethionate has a particle size D90 of 3-60 µm.
